(19) United States
(12) Patent Application Publication
(10) Pub. No.: US 2011/0166562 A1
(43) Pub. Date: Jul. 7, 2011

(54) High Durability Side Fire Optical Fiber for High Power Applications

(75) Inventors: Alexander Harschack, Bad Honnef (DE); Wolfgang Neuberger, Dubai (AE)

(73) Assignee: CeramOptec Industries, Inc.

(21) Appl. No.: 12/651,749

(22) Filed: Jan. 4, 2010

(51) Int. Cl. A61B 18/20; C03B 37/075; C03B 37/018

(52) U.S. Cl. 606/16; 65/397; 65/392

(57) ABSTRACT

An optical fiber treatment system is provided for high power laser transmission to an area of medical treatment. A side fire optical fiber tip for use in high power laser applications having outputs of greater than or equal to 50 Watts is a key to the system. Embodiments are particularly appropriate for the medical treatment of benign prostate hyperplasia (BPH) with a side fire optical fiber tip using a Holmium:YAG laser or a high power diode laser. Such procedures can be done with only local anesthesia. A predetermined length of an output tip on the distal end of the optical fiber of the present invention is formed with an optical fiber core and cladding layer of preselected thickness wherein the cladding to core diameter ratio is at least as great as 1.1. A side fire surface is formed on the distal end of the core/clad output end. Over this optical fiber output end, a pure silica capillary tube is fused to the predetermined length of exposed cladding where the outermost cladding is also pure silica to reduce thermal mismatch during the fusion process. By having the refractive index at the fusing interface of the tube to the cladding matched, and bubbles or gaps eliminated or prevented, it is possible to substantially enhance durability and eliminate Fresnel reflection losses at this interface. Further, the outer surface area where the laser energy is exiting from the tube may be heat treated with a laser to additionally increase durability for high power laser energy transmission in fluid environments. Germanium-doped silica as well as pure silica can be used as fiber core material.

### DOMESTIC PRIORITY UNDER 35 USC 119(e)

This application claims the benefit of U.S. Ser. No. 11/592,598, filed Nov. 3, 2006, entitled “Side Fire Optical Fiber for High Power Applications”, which is a continuation of U.S. Provisional Application Ser. No. 60/736,107, filed Nov. 10, 2005, and which are both incorporated by reference herein.

### BACKGROUND OF THE INVENTION

1. Field of the invention

The present invention relates generally to the field of the medical treatment using laser energy, and, further, relates to the transmission of high power laser energy over an optical fiber through an side-firing output end modified according to the present invention, and, more specifically, relates to the medical treatments with a highly durable side fire optical fiber tip.

2. Invention Disclosure Statement

Benign prostatic hyperplasia (BPH) or “enlarged prostate” refers to the non-cancerous (benign) growth of the prostate gland. While BPH is the most common prostate problem in men over 50 years of age, benign growth of the prostate begins with microscopic nodules around 25 years of age but rarely produces symptoms before a man reaches age 40. It is estimated that 6.3 million men in the United States alone have BPH and the disease is responsible for 6.4 million doctor visits and more than 400,000 hospitalizations per year.

The exact cause of BPH is unknown but it is generally thought to involve hormonal changes associated with the aging process. Testosterone likely has a role in BPH as it is continually produced throughout a man's lifetime and is a precursor to dihydrotestosterone (DHT) which induces rapid growth of the prostate gland during puberty and early adulthood. When fully developed, the prostate gland is approximately the size of a walnut and remains at this size until a man reaches his mid-forties. At this point the prostate begins a second period of growth which for many men often leads to BPH later in life.

In contrast to the overall enlargement of the gland during early adulthood, benign prostate growth occurs only in the central area of the gland called the transition zone, which wraps around the urethra. As this area of the prostate grows, the gland presses against the urethra and causes a number of lower urinary tract symptoms (LUTS) such as difficult urination (obstructive symptoms) and painful urination (storage symptoms). Eventually, the bladder itself weakens and loses the ability to empty itself.

Obstructive symptoms such as intermittent flow or hesitancy before urinating can severely reduce the volume of urine being eliminated from the body. If left untreated, acute urine retention can lead to other serious complications such as bladder stones, urinary tract infections, incontinence, and, in rare cases, bladder damage, kidney damage. These complications are more prevalent in older men who are also taking anti-arrhythmic drugs or anti-hypertensive (non-diuretic) medications. In addition to the physical problems associated with BPH, many men also experience anxiety and a reduced quality of life.

Mild symptoms of BPH are most often treated with medication such as alpha-blockers and anti-androgens. Men suffering with moderate to severe BPH symptoms typically must undergo surgery. Transurethral resection of the prostate (TURP) is the standard surgical procedure, although there are a number of other surgical approaches available as well. Other less invasive surgical methods include: transurethral incision of the prostate (TUIP), transurethral microwave thermotherapy (TUMT), transurethral electro vaporization (TUVP), transurethral needle ablation (TUNA), and laser surgery.

There are a number of different laser techniques in which light is used to eliminate excess prostate tissue either by ablation (vaporization) or thermal coagulation mechanisms. The observed clinical effects are due to the absorption of light (by the target tissue itself and/or surrounding fluids) and subsequent heat transfer, the extent of which largely depends on the power and wavelength of the laser beam. In general, wavelength determines the depth of tissue penetration while power has a direct influence on the temperature created within the tissue. However, it is temperature that determines the ultimate impact at the treatment area since tissue must be heated to greater than 50 C to induce coagulation whereas vaporization occurs at temperatures over 100 C. Temperature also impacts morbidity, namely inflammation, dysuria, bleeding, and the need and duration of post-treatment catheterization.

Laser approaches currently in use for the treatment of BPH utilize a single wavelength of light to eliminate excess prostate tissue via ablation or by inducing coagulation necrosis. Initially, however, laser surgeries for BPH used the Holmium:YAG laser in combination with the Nd:YAG laser in a treatment method called Combination Endoscopic Laser Prostatectomy (CELAP) being a two step process where the Holmium laser was used to create the channel through the prostate and the Nd laser was used for coagulation. It was further determined that the Nd laser was unnecessary if the Holmium laser was defocused for coagulation purposes. For the CELAP procedure, the Holmium laser was used to create a channel in the prostate by vaporizing the tissue after which, the Nd:YAG laser was used to further eliminate tissue via coagulation. CELAP has been replaced by newer, single-wavelength laser methods which are still being evaluated for long-term efficacy.

Holmium Laser Enucleation of the Prostate or HoLEP is a laser ablation technique in which a 2140 nm Ho:YAG laser is used to remove whole lobes from the prostate. Specifically, HoLEP uses a bare optical fiber which is brought into direct contact with the target tissue. Enucleation occurs when the vapor bubbles that form in front of the fiber bombard the target tissue and tear it apart. Special morcellators or other extraction techniques are needed to remove tissue debris from the area. The efficacy of the HoLEP procedure depends upon maintaining very close contact between the fiber and the tissue to be removed. As a result, it is possible to perforate the prostate during the procedure and many surgeons avoid using HoLEP because of the difficulty in learning and maintaining proficiency in the technique.

Another laser technique to eliminate prostate overgrowth features a frequency-doubled Nd:YAG laser as a pulsed, 532 nm high-power potassium-titanyl-phosphate laser (KTP) that vaporizes target tissue as well as induces thin layers of coagulation in the surrounding tissues as described by Davenport et aL in U.S. Pat. No. 6,554,824. The 532 nm radiation used in KTP is selectively absorbed by hemoglobin and penetrates tissues only to a depth of 1-2 mm. Moreover, this method requires continuous irrigation of the treatment site to cool the tissue during the procedure to help reduce unwanted coagulation necrosis in deeper tissue layers.

Much like TURP, most types of laser surgeries are able to provide an immediate improvement in the urinary stream. Laser surgery for BPH has other potential advantages such as reduced blood loss as well as shorter treatment times, faster patient recovery, and a lower risk of post-treatment incontinence. However, many patients still require catheterization for 1-2 weeks post-treatment after undergoing some forms of laser surgery. Despite the obvious benefits of laser procedures for BPH, long-term follow-up studies on the clinical results of many laser techniques are not yet widely available.

To date, none of the less invasive procedures have proven to be more effective than TURP nor are they generally appropriate across all patient groups including: younger men, debilitated elderly patients, patients with severe medical conditions including uncontrolled diabetes, cirrhosis, active alcoholism, obesity, and heart disease, as well as men taking blood thinning medications. As such, there remains a need for a device and treatment method for the effective alleviation of BPH symptoms that can be used across all patient groups with a minimum of adverse complications post-treatment. The present invention is directed towards this need.

U.S. Pat. No. 4,740,047 to Hitachi Cable et al. discusses the disadvantages of a side fire fiber where the fiber is placed in a transparent tubular member. As noted the several disadvantages are an air layer between the fiber and the tubular member resulting in leaking beams, multiple reflections, breakage of the tip, and in the use of this fiber tip in a front-view type of endoscope. The irradiation probe shown has a lateral beaming fiber with conventional cladding with an air space about the tip. The tubular member is attached to the fiber by means of several coatings of plastic material for the purpose of reducing direct external forces on the fiber. In order to prevent leaking beams, anti-reflective coatings are applied to the external surface of the tubular member in flat areas on the tubular member. The present invention reduces or eliminates these problems.

U.S. Pat. No. 5,292,320 to Brown et al. discloses another side firing output end having multiple side fire surfaces within the fiber core and is incorporated by reference. The fiber core has a plurality of grooves as well as a slanted end surface for reflecting laser energy in a lateral manner. The core is glued into the end cap. Under high power laser operations, for example, 50 W or greater, this output end fails.

U.S. Pat. No. 5,437,660 to Johnson et al. discloses a device for treating the body with a side fire feature thereon and is incorporated by reference. An end cap having a reflective surface therein is attached over the end of the fiber.

U.S. Pat. No. 5,509,917 to Cecchetti et al. discloses a lateral beaming laser tip having a transparent quartz cap about the output end of the optical fiber therein and is incorporated by reference. In contrast to the present invention, the tip has an extended section of optical core placed into the cap with an air gap about the core. Further, the cap is shown having various focusing means for the laser radiation reflected off of the slanted end surface of the optical core. This laser tip is very complex to manufacture and to achieve the very same structures each time.

U.S. Pat. No. 6,554,824 to Davenport et al. discloses a treatment means for a prostrate gland also having a side fire feature as noted in figures and is incorporated by reference. The actual construction of the tip is not disclosed.

U.S Pat. No. 6,802,838 to Loeb et al. discloses another side fire laser fiber enclosed within a tube with circulating fluid thereabout and is incorporated by reference. As noted the tube being the cap is placed over the bared distal end portion of the optical fiber by thermal fusion or to a buffer coat and vinyl cladding thereof by an adhesive.

There is thus a need for a laser treatment system that improves on the state of the art by allowing increased high power laser beam density transmission, high reliability of the output tip, and thereby providing increased power density available for medical treatments.

### OBJECTIVES AND SUMMARY OF THE INVENTION

It is an objective of the present invention to provide a system that increases the beneficial aspects of the treatment of BPH, in particular, with high power laser energy by increasing the damage threshold of the optical fiber tip and by increasing the reliability of the laser tip.

It is another objective of the present invention to provide for the efficient treatment of certain medical conditions with the use of high power laser energy to a treatment or work site while maintaining laser beam quality.

It is yet another objective of the present invention to provide a side fire optical fiber tip being able to handle a high power laser with the tip having excellent durability.

It is still a further objective of the present invention to provide a side fire optical fiber tip for use with high power lasers, from about 50 to about 400 Watt applications, having a durable tip and further presenting a minimum of manufacturing problems.

Briefly stated, the present invention provides an optical fiber treatment system for high power laser transmission to an area of medical treatment. A side fire optical fiber tip for use in high power laser applications having outputs of greater than or equal to 50 Watts is a key to the system. Embodiments are particularly appropriate for the medical treatment of benign prostate hyperplasia (BPH) with a side fire optical fiber tip using a Holmium:YAG laser or a high power diode laser. Such procedures can be done with only local anesthesia. A predetermined length of an output tip on the distal end of the optical fiber of the present invention is formed with an optical fiber core and cladding layer of preselected thickness wherein the cladding to core diameter ratio is at least as great as 1.1. A side fire surface is formed on the distal end of the core/clad output end. Over this optical fiber output end, a pure silica capillary tube is fused to the predetermined length of exposed cladding where the outermost cladding is also pure silica to reduce thermal mismatch during the fusion process. By having the refractive index at the fusing interface of the tube to the cladding matched, and bubbles or gaps eliminated or prevented, it is possible to substantially enhance durability and eliminate Fresnel reflection losses at this interface. Further, the outer surface area where the laser energy is exiting from the tube may be heat treated with a laser to additionally increase durability for high power laser energy transmission in fluid environments. Germanium-doped silica as well as pure silica can be used as fiber core material.

The above, and other objects, features and advantages of the present invention will become apparent from the following description read in conjunction with the accompanying drawings, (in which like reference numbers in different drawings designate the same elements).

### BRIEF DESCRIPTION OF DRAWINGS

FIG. 1 illustrates by block diagram of a medical laser device using the present invention in the treatment of BPH.

FIG. 2 illustrates an optical fiber having an output end being a fiber core with one cladding layer with a side fire surface thereon.

FIG. 3 illustrates by cross sectional view an optical fiber output tip of the present invention.

FIG. 4 illustrates, by way of example, the indices of refraction of the core, clad and tube sections, respectively, of the present invention.

### DETAILED DESCRIPTION OF PREFERRED EMBODIMENTS

The present invention describes an optical fiber having a side fire tip that is useful for high power laser beam transmission through an optical fiber. The side fire tip is used in medical treatment and has high reliability and durability. The present invention provides a side fire optical tip for high power lasers, in particular, having outputs of greater than or equal to 50 Watts to about 400 Watts. The present invention is particularly appropriate for the medical treatment of benign prostate hyperplasia (BPH) with a side fire optical fiber tip with a Holmium:YAG laser.

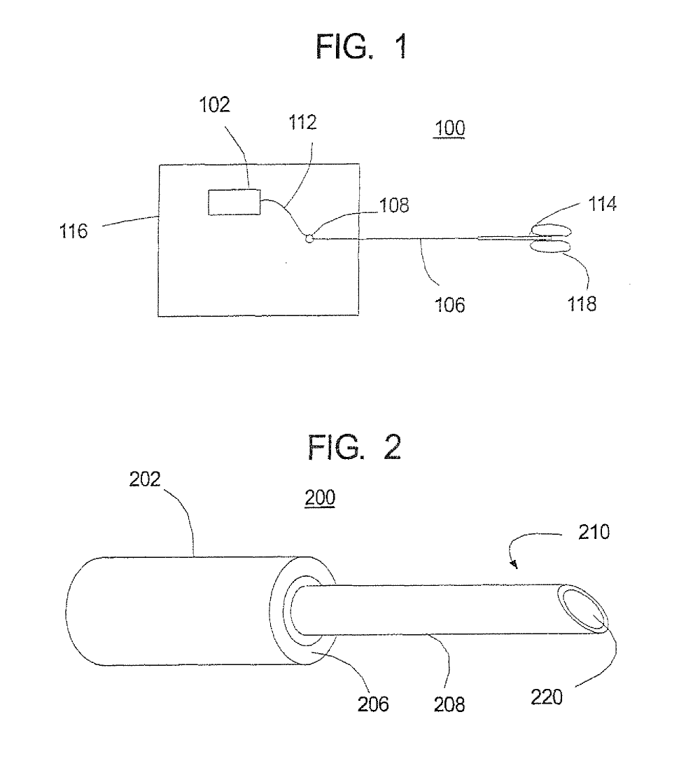

FIG. 1 is a block diagram depicting one embodiment of medical laser device 100 in accord with the present invention. Device 100 includes one laser source. The preferred laser for the present invention is a Holmium:YAG laser, but other laser sources may be included such as a 980 nm diode laser, 1470 nm diode laser or a 1950 nm diode laser. Laser source 102 outputs radiation into optical fiber 112. Connector 108 takes the input from optical fiber 112 and outputs the laser energy into output optical fiber 106 that delivers the radiation into output end 114. Device 100 further includes control device 116 where the operational parameters are input. These operational parameters would include power, duration, repetition rate, and continuous or pulse mode, energy density, etc., for the laser therein. Output end 114 is shown within prostrate gland 118. A suitable catheter or endoscope device is used to place output end 114 with attached optical fiber 106 in the urethra up to the area of the surrounding prostrate tissue of concern. Further other devices may be included in the catheter such as viewing means, irrigating means, cooling means, etc.

In a typical mode of operation, output end 114 irradiates the target area with a preselected pattern of pulses and energy densities. Treatment can also involve a semi-continuous irradiation for each position to be treated, or with a turning of the side firing fiber probe to circumferentially treat a larger section. In preferred embodiments, these patterns of pulses and energy result in the ablation of prostatic tissue as well as coagulation of underlying tissues to substantially eliminate blood loss beyond the removed tissue and with minimal thermal damage to deeper and surrounding tissue.

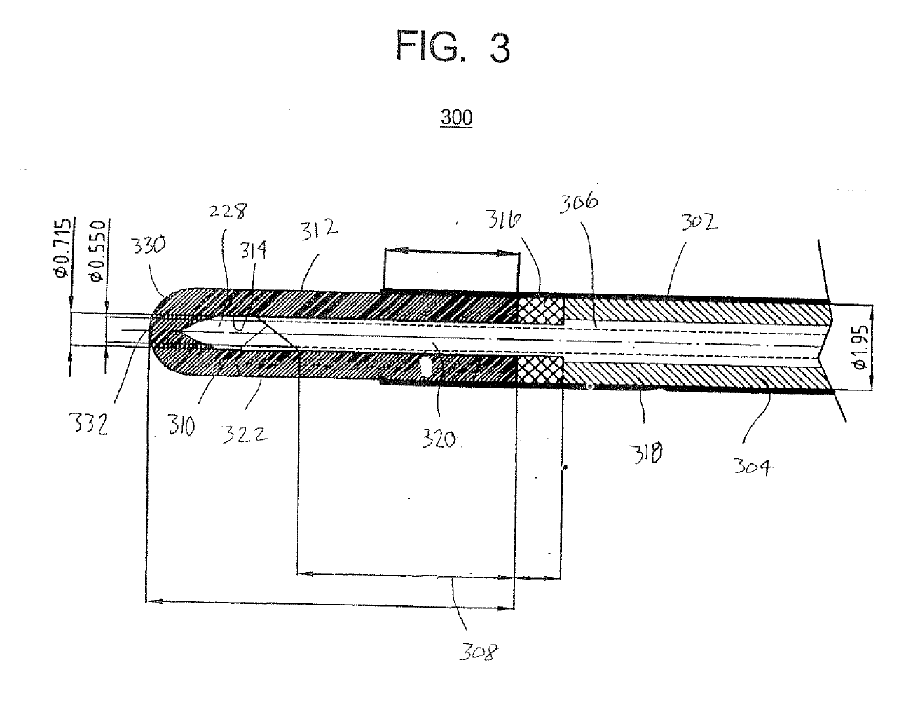

FIG. 2 depicts side fire optical fiber 200 with an output end 210 that may be used in the method of the present invention. Output end 210 has side fire surface 220 that is slanted at a given angle to the axis of optical fiber 202. A reflective coating may optionally be included on the surface 220. A predetermined length of optical fiber 200 has outer protective layer 206 removed to leave cladding layer 208 and core as shown. In the preferred embodiment, cladding layer 208 is composed of low OH, pure silica. Cladding material is originally deposited by a preferred method of plasma enhanced deposition on an outer surface of core material to make a preform(not shown) which is drawn to produce optical fiber 200. During the deposition process the percentage of fluorine is reduced so that the material is low OH, pure silica further from core 212 than at the core/cladding interface After drawing of the fiber, capillary tube 312 (FIG. 3) is fused to cladding layer 208 as will be disclosed hereinafter.

FIG. 3 illustrates optical fiber output tip 300 having a side fire feature. Optical fiber 302 being of conventional design has a protective or buffer layer 304 removed to leave cladding layer 306 on output end 308. On a distal end of output end 308, side fire surface 310 is formed and is of conventional design. The slant angle itself may range from 30 to 50 degrees as is conventional. Due to the diameter of the optical fiber, multi-mode reflections at side fire surface 310 provide an output beam that is somewhat projecting forward and is spreading out as it leaves output tip 300 at a range of angles, typically 70-100 degrees from the fiber axis . To further enhance the reflective power of surface 310 a reflective layer may be formed thereon, as is known in the optical arts.

Onto output end 308, capillary tube 312 having inside hole 314 being of close fit about output end 308 is fused to outer most surface region of clad layer 306. A CO₂ laser may be used to fuse tube 312 to output end 308. This fusing process, is performed in such a way that bubbles or air gaps do not exist between the clad layer 306 and tube 312. Bonding section 316 secures tube 312 to protective layer 304 of optical fiber 302 to prevent undue strain on output end 308. Shrink tube 318 further protects the output tip 300 and is placed over section of tube 312, bonding section 316 and optical fiber 302.

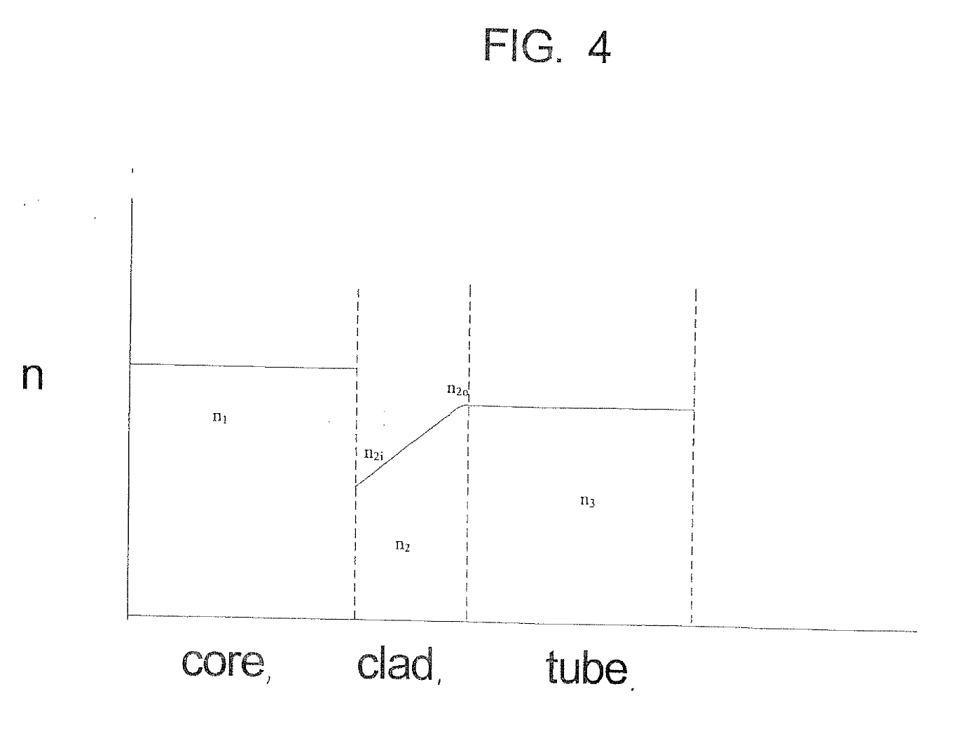

Core 320 in the output section 308 is composed of silica having an index of refraction of n₁; clad layer 306 is also composed of silica and has an index of refraction of n₂ which at the interface with the core is less than n₁ , but increases toward the outward edge of the cladding to a value equal to pure silica. Reference is made to FIG. 4 that shows a relationship between the indexes of refraction in the different sections of the output end 308. In this scheme, the core material 320 can be pure silica or a Germanium doped silica. The cladding material 306 would be a fluorine-doped silica. Further in the case of the doped core, the cladding material could be all pure silica too. In the latter case the cladding would have a uniform refractive index across its thickness that would match the refractive index the tubing material 312 as the tube is also pure silica as noted throughout this specification.

To insure a minimum of reflection losses and enhanced durability at the clad/tube interface, refractive indices are matched and care is taken to not permit formation of bubbles and air gaps at the interface between clad 306 and tube 312 during the fusion process. Capillary tube 312 is also composed of silica and has an index of refraction of n₃. The silica at the outer edge of clad layer 306 and in tube 312 is preferably pure silica, that is having the same refractive index, n₃. During the deposition of clad layer 306, when it is a fluorine-doped silica, the amount of fluorine is reduced so that at the fusing interface, clad layer is low OH, pure silica, and has essentially the same index of refraction as tube 312, also composed of low OH, pure silica.

To insure that the clad at the interface and the tube are essentially continuous mechanically and optically after fusing, the thermal behavior of the two facing surfaces shall be as equivalent as possible. Matching refractive indices is one measure to achieve this equivalence. Since glass is a viscous liquid its manufacturing process can also affect its thermodynamic properties, thus in a further embodiment, the manufacturing process of the tube can be made essentially the same as the outer surface layer of the cladding. That is, the thermal history of the outermost layer of the cladding is substantially equivalent to the theinial history of the inner surface of the tube. Alternatively, one can say that the fictive temperature of the opposing surfaces are equivalent, i.e. the fictive temperature of the outermost portion of clad layer 306, is essentially equivalent to the fictive temperature of the innermost portion of tube 312.

In an additional preferred embodiment an even more robust side firing distal end can be produced by further processing. The laser energy output area, being surface 322, is heat treated to create a more durable surface to high power laser energy and for mechanical properties. Surface 322 may be heat treated by a laser beam such as a CO₂ laser provides.

Sealed end section 330 is a distal section of capillary tube 312. The distal section is heated and closed to form sealed end section 330. Front tip 332 is rounded and preferably heat treated.

The present invention is further illustrated by the following examples, but is not limited thereby:

### EXAMPLE 1

In accord with the present invention, a medical laser with a maximum average power of 150 Watts and having an output wavelength of 2150 nm can be used in the treatment of BPH. Lasing can be performed using a 550 μm core with cladding layer 306 of 715 μm diameter, with a cladding to core ratio of 1.3. Optical fiber 302 has a diameter of 1950 μm, for example. Optical fiber output tip 300 can be positioned close to the target tissues such as a prostrate gland by a rigid endoscope, for example, with water as an irrigant to further cool the tip and help remove material as it is ablated, or degraded during high power operation.

Preferred embodiments may also be used to transmit high power laser energy from diode lasers operating at 980 nm, 1460 rim, 1900 nm or combinations with individual or combined power levels from 50 to about 400 Watts. Using fibers of similar size to that described above various medical treatments including treatment of BPH can be done very effectively, quickly and without requiring more than local anesthesia for the patient.

In a typical mode of operation, from FIG. 1, optical fiber 106 delivers laser energy from laser source 102 to a handpiece, endoscope, or similar instrument for positioning the fiber's distal output end 114 in close proximity, in direct contact with, or inside/within the target tissue. It is preferred that the handpiece, endoscope, or similar instrument have sufficient channels to accommodate the flow and removal of irrigant and/or debris from the treatment site, endoscopic instruments, aspirators, light guides, image guides, or other sensor and/or detection means. Benefits from using, for example, a 980 nm diode laser, instead of a Ho:YAG laser, are somewhat greater depth penetration, and photocoagulation leading to a substantially blood free operating area.

Having described preferred embodiments of the invention with reference to the accompanying drawings, it is to be understood that the invention is not limited to the precise embodiments, and that various changes and modifications may be effected therein by one skilled in the art without departing from the scope or spirit of the invention according to the appended claims.

What is claimed is:

1. An optical fiber treatment system having a side fire output tip for transmitting high power laser energy in medical treatments, said output tip comprising:
an optical fiber, having a core and a cladding, for transmitting high power laser energy, said optical fiber also having an input end and an output end;
said output end of said optical fiber being of a predetermined length, said output end being a fiber core and a clad layer over said predetermined length, said output end having a side firing surface formed thereon;
said clad layer having a variable index of refraction in a radial direction;
a capillary tube, said capillary tube for attachment to said output end on an outer most surface of said clad, said capillary tube having a hole at a proximal end for the acceptance of said output end of said optical fiber, having a sealed end section being formed from a distal end section of said capillary tube, and also having an output area therein where said laser energy passes through said tube;
wherein at said clad interface with said core, said variable index is lower than the refractive index of said core and at said clad outer surface, said variable index is equivalent to a refractive index of an innermost surface of said capillary tube in contact with said clad at a clad-tube interface;
a void, said void being located between said side fire surface and said sealed end section,
wherein said capillary tube is fused to said outer most surface of said clad layer along the predetermined length of said clad layer,
wherein said refractive indices at said fusing interface of said tube and of said outer surface area of said clad are matched to minimize/eliminate thermal mismatches at this interface; and
wherein said clad-tube interface is free of gaps or bubbles, thereby producing a highly durable distal tip.

2. The optical fiber output tip according to claim 1, wherein said high power laser energy is from about 50 to about 400 Watts.

3. The optical fiber output tip according to claim 1 wherein at said clad outer surface area and said tube inner surface are composed of low OH, pure silica.

4. The optical fiber output tip according to claim 3 wherein a fusing interface of said tube and said cladding layer are thermally matched surfaces, having equivalent fictive temperatures.

5. The optical fiber output tip according to claim 1, wherein said fiber core is germanium-doped silica and said clad is pure low OH silica, and wherein said cladding refractive index profile is essentially uniform radially and equal to said refractive index of said inner surface of said tube.

6. The optical fiber output tip according to claim 1 wherein said laser energy is selected from the group consisting of a Holmium:YAG laser, a 980 nm diode laser, a 1460 nm diode laser and a 1900 nm laser.

7. The optical fiber output tip according to claim 1 wherein said output area is heat treated with a CO₂ laser.

8. The optical fiber output tip according to claim 1 wherein the clad diameter to core diameter ratio (clad/core ratio) is from about 1.1 to about 1.4.

9. A method of minimizing thermal mismatching in an output end of a side fire optical fiber tip to provide highly durable tips comprising the steps of:
forming an optical fiber wherein said optical fiber has a cladding layer at an output end, a cladding to core diameter ratio being at least 1.1;
when a fluorine-doped clad is used, reducing percentage of fluorine in said cladding layer during deposition to match the refractive indices of said cladding to a capillary tube, while maintaining a desired index difference at a clad-core interface;
creating an angled surface at a distal end of said optical fiber;
slipping said capillary tube over said cladding layer;
fusing said capillary tube to a predetermined length of cladding wherein gaps and bubbles over said predetermined fused length are eliminated; and
sealing a distal end of said tubing into a rounded surface to provide an air gap between said angled surface of said optical fiber and said rounded surface.

10. The method according to claim 9, having a further step of:
heat treating said fused distal end with a laser to further improve optical and mechanical performance.

11. The method according to claim 9, wherein at least an outer most layer of said cladding and said tube are composed of low OH, pure silica, having essentially the same index of refraction, and thermal properties, including thermal histories and fictive temperatures.

12. The method according to claim 9, wherein said core material is composed of germanium-doped silica, and said cladding is composed of low OH, pure silica.

13. The method according to claim 9, wherein said cladding is deposited on said core by means of a plasma enhanced deposition process.

14. The method according to claim 9, wherein said clad to core diameter ratio is at least 1.2.

15. The method according to claim 10, wherein said fusing step and said heat treating step are performed with a CO₂ laser.
